(19) United States
(12) Patent Application Publication
(10) Pub. No.: US 2012/0219943 A1
(43) Pub. Date: Aug. 30, 2012

(54) METHODS OF PROGNOSIS AND DIAGNOSIS IN CHRONIC HEART FAILURE

(75) Inventors: Bonnie Ky, Philadelphia, PA (US); Thomas Peter Cappola, Haverford, PA (US)

(73) Assignee: ABBOTT LABORATORIES, Abbott Park, IL (US)

(21) Appl. No.: 13/356,935

(22) Filed: Jan. 24, 2012

Related U.S. Application Data
(60) Provisional application No. 61/439,227, filed on Feb. 3, 2011.

(51) Int. Cl. C12Q 1/68; G01N 33/566

(52) U.S. Cl. 435/6.11; 435/6.12; 435/7.1; 435/7.92; 435/7.93; 435/7.94; 435/7.95; 436/501

(57) ABSTRACT

The present disclosure provides methods of diagnosing chronic heart failure in patients by detecting the presence and amounts of biomarkers of heart failure in samples from the patients. Such biomarkers may be used to develop a more accurate prognosis for a patient with heart failure, or to accurately diagnose a patient suspected of having heart failure.

### RELATED APPLICATION INFORMATION

This application claims priority to U.S. Provisional Application Ser. No. 61/439,227, filed on Feb. 3, 2011, the contents of which are herein incorporated by reference.

### BACKGROUND OF THE INVENTION

1. Technical Field

The subject invention relates to methods for determining prognosis or risk stratification for chronic heart failure patients by detecting particular biomarkers in the patients as well as amounts thereof. Such biomarkers may be used to accurately develop a prognosis for a patient with heart failure, to stratify risk in heart failure patients, and also to develop a diagnosis.

2. Background

Although heart failure is primarily a disorder of the myocardium, abnormal vascular function has a major impact on heart failure progression and cardiac remodeling. Angiogenic growth factors, such as the vascular endothelial growth factor (VEGF) family of proteins, govern numerous aspects of vessel homeostasis in coronary and peripheral vascular beds. In the setting of ischemic heart disease, alterations in angiogenic growth factors contribute to endothelial cell dysfunction and impaired revascularization. Moreover, even in the setting of nonischemic heart disease, angiogenic factors regulate myocardial capillary density as the heart hypertrophies, exert antiapoptoic and protrophic effects in dilated cardiomyopathy, and influence peripheral vascular load.

Placental growth factor (PlGF) is a member of the VEGF family of angiogenic proteins and is expressed in placental, cardiac, and lung tissue. PlGF activates the Fms-like tyrosine kinase receptor 1 (Flt-1) and is expressed in numerous cell types including endothelial cells, monocytes and renal mesangial cells. The Flt-1 receptor has affinity for PlGF, VEGF-A, and VEGF-B. In animal models, PlGF/Flt-1 signaling exerts pleiotrophic effects. These include potentially beneficial effects, such as the promotion of angiogenesis, and potentially harmful proinflammatory effects that may contribute to atherogenesis. Consequently, the overall effect of PlGF/Flt-1 signaling in cardiovascular disorders is difficult to predict and may vary according to disease state and comorbid conditions.

To better understand PlGF/Flt-1 signaling in the setting of human disease, scientists have capitalized on the observations that both PlGF and the circulating form of the Flt-1 receptor, soluble Flt-1 (sFlt-1) can be easily quantified. Although there are biochemical interactions between PlGF and sFlt-1 in peripheral plasma, the levels of both factors can provide a method to conveniently gauge overall PlGF/sFlt-1 activity in patients with cardiovascular disorders. During pregnancy, changes in circulating PlGF and sFlt-1 reflect endothelial function and predict preeclamptic risk. In patients with chest pain and acute coronary syndromes, higher PlGF levels are seen in those with myocardial infarction and are associated with an increased risk of short and long-term adverse outcomes. Studies of circulating sFlt-1 have demonstrated conflicting results, with some studies noting higher levels during acute myocardial infarction (MI) compared to control patients, and others noting lower plasma levels in patients during the acute phase of MI compared to controls.

Although PlGF and sFlt-1 may be important disease modifiers, neither factor has been comprehensively studied in chronic human heart failure. The largest published experience on PlGF was a cross-sectional study of 98 patients that showed a positive relationship between PlGF levels and New York Heart Association (NYHA) class in ischemic heart failure, but not in nonischemic disease (T. Nakamura et al., Elevation of plasma placental growth factor in the patients with ischemic cardiomyopathy. INT J CARDIOL. 131:186-91 (2009)). Circulating sFlt-1 has not been studied in chronic human heart failure.

Thus, in view of the above, significant need exists to study PlGF and sFlt-1 and determine their value as clinical markers for chronic heart failure. If valuable, patients diagnosed with chronic heart failure may then be treated immediately by physicians, thereby potentially reducing the incidence of fatality from this serious condition. Further, a need exists for such biomarkers that can be used for prognostic or predictive purposes to determine if a patient will likely experience a particular outcome.

### SUMMARY OF THE INVENTION

In one aspect, the present disclosure provides a method for providing a diagnosis, prognosis or risk classification of a subject having or at risk of having heart failure, the method comprising: a) providing a biological sample from the subject; b) determining the concentration of soluble Flt-1 (sFlt-1) in the sample; and c) comparing the determined sFlt-1 concentration with a reference sFlt-1 value, wherein a determined sFlt-1 concentration of the subject greater than the reference sFlt-1 value is indicative of heart failure or increased risk of heart failure in the subject. The method may further comprise assessing at least one additional biomarker of heart failure. In the method, providing a diagnosis can be providing a diagnosis of heart failure. Alternatively, providing a prognosis can be determining heart failure severity, or can be risk assessment of the subject with heart failure. In the method, heart failure can be chronic heart failure, systolic heart failure, dilated cardiomyopathy (DCM), ischemic cardiomyopathy, acute myocardial infarction, left ventricular dysfunction, or right ventricular dysfunction. The method may further comprise the assessment of at least one additional biomarker of heart failure selected from B-type natriuretic peptide (BNP), NT-pro-BNP, pro-BNP, creatinine, PAPP-A, cardiac troponin I (TnI), cardiac troponin T (TnT), neuregulin-1, VEGF, PlGF, soluble CD40 ligand (sCD40L), myeloperoxidase (MPO), growth-differentiation factor 15 (GDF-15), soluble ST-2 protein (also known as IL1RL1), copeptin (C-terminal provasopressin), adrenomedullin, high sensitivity C-reactive protein (hs-CRP), uric acid, and galectin-3 (gal-3). Assessment of the additional biomarker may comprise, for example, measuring the concentration of the biomarker in the biological sample from the subject, or may comprise a clinical evaluation of the subject. For an additional biomarker assessed by measuring the concentration of the biomarker in the biological sample from the subject, the method may further comprise comparing the measured concentration of the at least one further biomarker with a reference value for the biomarker. The reference value for the additional biomarker can be the biomarker concentration of a control sample, a biomarker cut-off value, or a median concentration of a plurality of control samples from a group of control subjects. The additional biomarker can be, for example, BNP, and the reference value for BNP can be a median value of about 177 pg/ml in plasma.

In another aspect, the present disclosure provides a method for identifying one or more patients or a subgroup of patients having an increased cardiac risk, the method comprising: a) providing a biological sample from at least one patient having or suspected of having an increased cardiac risk compared to a reference cardiac risk; b) determining the concentration of soluble Flt-1 (sFlt-1) in the sample, and c) comparing the determined sFlt-1 concentration with at least one reference value, wherein a determined concentration of sFlt-1 greater than the reference value is indicative of increased cardiac risk of the patient. The method may further comprise assessing at least one additional biomarker of increased cardiac risk. The reference value for the additional biomarker can be the biomarker concentration of a control sample, a biomarker cut-off value, or a median concentration of a plurality of control samples from a group of control subjects. The additional biomarker can be, for example, BNP, and the reference value for BNP can be a median value of about 177 pg/ml in plasma.

In another aspect, the present disclosure provides a method for diagnosis, prognosis and/or risk stratification of cardiovascular disease in a subject having or suspected of having heart failure, the method comprising the detection of an increased sFlt-1 concentration in the subject.

In another aspect, the present disclosure provides a method for providing a diagnosis or prognosis of a subject having or at risk of having renal disease, the method comprising: a) providing a biological sample from at least one patient having or at risk of having renal disease; b) determining the concentration of soluble Flt-1 (sFlt-1) in the sample, and c) comparing the determined sFlt-1 concentration with at least one reference value, wherein a determined concentration of sFlt-1 greater than the reference value is indicative of renal disease in the patient. The method may further determining an estimated glomerular filtration rate (eGFR) in the subject, and comparing the determined eGFR with a reference eGFR value, wherein a determined concentration of eGFR greater than the reference eGFR value is further indicative of renal disease in the patient. In the method, the reference value may be the eGFR of a control subject or the median eGFR as determined from a group of control subjects.

In any of the methods, the sFlt-1 reference value can be the sFlt-1 concentration of a control sample or a sFlt-1 cut-off value. The sFlt-1 concentration can be for example the sFlt-1 plasma concentration. The control sample can be a biological sample of a control subject or an sFlt-1 standard. The sFlt-1 concentration of a control sample can be, for example, the median sFlt-1 concentration of a plurality of control samples from a group of control subjects. Alternatively, an sFlt-1 cut-off value can be determined by a receiver operating curve (ROC) analysis from biological samples of a patient group. Alternatively, an sFlt-1 cut-off value can be determined by a quartile analysis of biological samples of a patient group. For example, an sFlt-1 cut-off value can be determined by selecting a value that corresponds to the median of a patient group consisting of patients with chronic heart failure, which can be for example about 308 pg/ml plasma. Alternatively, an sFlt-1 cut-off value can be determined by selecting a value that corresponds to the 75^{th} percentile of a patient group consisting of patients with chronic heart failure, which can be for example about 380 pg/ml plasma.

In any of the methods, the subject can be a human subject and the biological sample of the subject and/or the control sample can be taken from a human subject. In any of the methods, the biological sample can be a bodily fluid, including any one of whole blood, plasma, serum, urine or any cell culture suspension or fraction of any thereof. In an exemplary method, the sample is whole blood, plasma or serum, preferably plasma. A coagulation inhibitor can be added to any peripheral blood sample. In the method, determining the concentration of sFlt-1, and optionally the at least one additional biomarker, can be performed by an immunological assay method in which a reagent capable of specific binding to sFlt-1, and optionally a reagent capable of specific binding to the additional biomarker, are used.

The present disclosure also provides a kit for performing any of the methods disclosed herein, wherein the kit includes at least one reagent capable of specifically binding sFlt-1, to quantify the sFlt-1 concentration in a biological sample of a subject, and a reference standard indicating a reference sFlt-1 concentration. In a kit for performing a method for providing a diagnosis, prognosis or risk classification of a subject having or at risk of having heart failure, the kit may further comprise at least one additional reagent capable of specifically binding at least one additional biomarker of heart failure in the biological sample, to quantify the concentration of the at least one additional biomarker in the biological sample, and also a reference standard indicating a reference concentration of the at least one additional biomarker of heart failure in the biological sample. In a kit for performing a method for providing a diagnosis or prognosis of a subject having or at risk of having renal disease, the kit may further comprise at least one additional reagent capable of specifically binding at least one additional biomarker of renal disease in the biological sample to quantify the concentration of the at least one additional biomarker in the biological sample, and a reference standard indicating a reference concentration of the at least one additional biomarker of renal disease in the biological sample. In any of the kits, the at least one reagent capable of specifically binding sFlt-1 may comprise at least one antibody capable of specifically binding sFlt-1.

### BRIEF DESCRIPTION OF THE DRAWINGS

FIG. 1A illustrates transplant-free and ventricular assist device (VAD)-free survival according to baseline levels of sFlt-1, as determined from Kaplan-Meier plots;

FIG. 1B illustrates transplant-free and ventricular assist device (VAD)-free survival according to baseline levels of PlGF, as determined from Kaplan-Meier plots; and

FIG. 2 illustrates ROC curves for baseline sFlt-1 and BNP at 1 Year of follow-up.

### DETAILED DESCRIPTION

The present disclosure is based on the unexpected discovery of a strong and independent association between sFlt-1 and chronic heart failure. In particular, the present disclosure discloses for the first time an association between sFlt-1 levels, NYHA Class, and risk of adverse outcomes in heart failure, and the further discovery that combined use of sFlt-1 level and the existing standard BNP is superior in classifying risk than use of either biomarker alone. As described herein, sFlt-1 is significantly associated with disease severity and clinical outcomes in chronic heart failure, and the association is robust, surviving even after adjusting for numerous confounding variables. Assessment of sFlt-1 can therefore improve on current ability to stratify patient risk and develop a prognosis in patients, thereby significantly benefiting patients having or at risk of developing chronic heart failure. Further, combined use of sFlt-1 and BNP can provide comparable advantages. The present disclosure further encompasses the similarly unexpected finding that circulating levels of sFlt-1 can vary according to renal function, with greater relevance in the setting of eGFR values within normal range. Thus, sFlt-1 is identified as a novel clinical biomarker of adverse outcomes in chronic heart failure, and is useful as such across the spectrum of ischemic and nonischemic disease.

Accordingly, the present disclosure provides methods of providing a diagnosis, prognosis or risk classification of a subject or group of subjects having or at risk of having heart failure, using sFlt-1 as a novel clinical biomarker of adverse outcomes. The present disclosure also provides methods of methods for providing a diagnosis or prognosis of a subject having or at risk of having renal disease, using sFlt-1 as a novel clinical biomarker of renal disease, particularly in cases where eGFR values are within normal range. Also provided are kits for performing the disclosed methods.

Section headings as used in this section and the entire disclosure herein are not intended to be limiting.

### A. DEFINITIONS

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

The use of “or” means “and/or” unless stated otherwise. Furthermore, the use of the term “including”, as well as other forms, such as “includes” and “included”, is not limiting.

As used herein, the term “sFlt-1” refers to the circulating form of the Flt-1 receptor, also referred to as soluble fms-like tyrosine kinase-1, soluble Flt-1 and sVEGFR-1, which is a tyrosine kinase protein that specifically binds free circulating VEGF (vascular endothelial growth factor) and PlGF (placental growth factor).

As used herein, the phrase “heart failure” refers to a condition in which the heart cannot pump blood efficiently to the rest of the body. Heart failure may be due to damage to the heart or narrowing of the arteries due to infarction, cardiomyopathy (primary or secondary), hypertension, coronary artery disease, valve disease, birth defects or infection. Heart failure can further be described as chronic, congestive, acute, decompensated, systolic or diastolic. The New York Heart Association (NYHA) classification describes the severity of the disease based on functional capacity of the patient; NYHA class can progress and/or regress based on treatment or lack of response to treatment. In heart failure, “increased severity” of cardiovascular disease refers to the worsening of disease as indicated by increased NYHA classification, to, for example, Class III or Class IV, and “reduced severity” of cardiovascular disease refers to an improvement of the disease as indicated by reduced NYHA classification, from, for example, class III or IV to class II or I.

The term “cardiomyopathy” refers to a weakening of the heart muscle or a change in heart muscle structure. It is often associated with inadequate heart pumping or other heart function abnormalities. Cardiomyopathy can be caused by viral infections, heart attacks, alcoholism, long-term, severe high blood pressure, nutritional deficiencies (particularly selenium, thiamine, and L-carnitine), systemic lupus erythematosus, celiac disease, and end-stage kidney disease. Types of cardiomyopathy include dilated cardiomyopathy, hypertrophic cardiomyopathy, and restrictive cardiomyopathy.

As used herein, the term “dilated cardiomyopathy” refers to a global, usually idiopathic, myocardial disorder characterized by a marked enlargement and inadequate function of the left ventricle. Dilated cardiomyopathy includes ischemic cardiomyopathy, idiopathic cardiomyopathy, hypertensive cardiomyopathy, infectious cardiomyopathy, alcoholic cardiomyopathy, toxic cardiomyopathy, and peripartum cardiomyopathy.

As used herein, the term “hypertrophic cardiomyopathy” refers to a condition resulting from the right and left heart muscles growing to be different sizes.

As used herein, the term “restrictive cardiomyopathy” refers to a condition characterized by the heart muscle's inability to relax between contractions, which inability prevents it from filling sufficiently.

The term “ischemic heart disease” refers to any condition in which heart muscle is damaged or works inefficiently because of an absence or relative deficiency of its blood supply; most often caused by atherosclerosis, it includes angina pectoris, acute myocardial infarction, and chronic ischemic heart disease.

“Angina pectoris” refers to chest discomfort caused by inadequate blood flow through the blood vessels (coronary vessels) of the myocardium.

A “myocardial infarction” (heart attack) occurs when an area of heart muscle dies or is damaged because of an inadequate supply of oxygen to that area.

As used herein, the phrase “clinical indicia” refers to assays, test methods (such as imaging), standards (such as The New York Heart Association (NYHA) classification), biophysical measures (such as LDL concentration, HDL concentration, triglyceride concentration, blood pressure, body mass index, waist circumference, heart rate, fasting insulin concentration, fasting glucose concentration, diabetes status) and other biometric parameters (such as, but not limited to, race, gender, age, tobacco smoking status, previous history of cardiovascular disease, family history of cardiovascular disease, use of high blood pressure medication etc.) that provide an indicator of cardiovascular disease.

As used herein, the terms “risk assessment” or “risk stratification” of subjects refers to the evaluation of factors including biomarkers, to predict the risk of occurrence of future events including death, so that treatment decisions regarding the subject may be made on a more informed basis.

As used herein, the term “cardiac risk” of subjects refers to the evaluation of factors including biomarkers, to predict the risk of occurrence of future heart failure events including increased probability of heart failure in any form, and death due to heart failure.

As used herein, the terms “specific binding” or “specifically binding”, refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “antibody” refers to an immunoglobulin molecule or immunologically active portion thereof, namely, an antigen-binding portion. Examples of immunologically active portions of immunoglobulin molecules include F(ab) and F(ab′) 2 fragments which can be generated by treating an antibody with an enzyme, such as pepsin. Examples of antibodies that can be used in the present disclosure include, but are not limited to, polyclonal antibodies, monoclonal antibodies, chimeric antibodies, human antibodies, humanized antibodies, recombinant antibodies, single-chain Fvs (“scFv”), an affinity maturated antibody, single chain antibodies, single domain antibodies, F(ab) fragments, F(ab′) fragments, disulfide-linked Fvs (“sdFv”), and antiidiotypic (“anti-Id”) antibodies and functionally active epitope-binding fragments of any of the above.

As used herein, the terms “subject” and “patient” are used interchangeably irrespective of whether the subject has or is currently undergoing any form of treatment. As used herein, the terms “subject” and “subjects” refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc) and a human). Preferably, the subject is a human.

The terms “sample” and “biological sample” as used herein generally refer to a biological material being tested for and/or suspected of containing an analyte of interest such as sFlt-1. The biological material may be derived from any biological source but preferably is a biological fluid likely to contain sFlt-1. Examples of biological materials include, but are not limited to, stool, whole blood, serum, plasma, red blood cells, platelets, interstitial fluid, saliva, ocular lens fluid, cerebral spinal fluid, sweat, urine, ascites fluid, mucous, nasal fluid, sputum, synovial fluid, peritoneal fluid, vaginal fluid, menses, amniotic fluid, semen, soil, etc. The sample may be used directly as obtained from the biological source or following a pretreatment to modify the character of the sample. For example, such pretreatment may include preparing plasma from blood, diluting viscous fluids, adding an anticoagulation factor such as heparin or EDTA, and so forth. Methods of pretreatment may also involve filtration, precipitation, dilution, distillation, mixing, concentration, inactivation of interfering components, the addition of reagents, lysing, etc. If such methods of pretreatment are employed with respect to the sample, such pretreatment methods are such that sFlt-1, and any other biomarkers such as BNP, remain in the sample at a concentration proportional to that in an untreated test sample (e.g., namely, a sample that is not subjected to any such pretreatment method(s)).

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### B. METHODS

The methods encompass providing a prognosis of a subject which includes, with respect to heart failure, any one or more of determining the severity of heart failure, determining the subject's risk for subsequent all-cause mortality, transplantation, or left ventricular assist device implantation, and risk assessment of the subject with heart failure. The methods are based in part on the novel finding that sFlt-1 concentration in a biological sample from a subject with heart failure predicts adverse outcomes of the subject, and thus that sFlt-1 is a prognosis marker for heart failure. For example, the sFlt-1 concentration of a subject can be used to provide a prognosis with respect to the risk for subsequent all-cause mortality, death, or cardiac transplantation, such as for example a mortality prediction within a given number of years.

The methods involve providing or obtaining a biological sample from the subject, which can be obtained by any known means including needle stick, needle biopsy, swab, and the like. In an exemplary method, the biological sample is a blood sample, preferably a blood plasma sample, which may be obtained for example by venipuncture. Biological samples may be or have been stored or banked under suitable tissue storage conditions.

The methods encompass a method for diagnosis, prognosis and/or risk stratification of cardiovascular disease in a subject having or suspected of having heart failure by determining an sFlt-1 concentration in the subject. Providing a diagnosis can be, for example, providing a diagnosis of heart failure. Providing a prognosis can be, for example, determining heart failure severity, or can be a risk assessment, i.e. determination of cardiac risk of the subject. The methods also encompass identifying one or more patients or a subgroup of patients having an increased cardiac risk. The methods also encompass providing a diagnosis of renal failure. A shared feature of all methods is the determination of concentration of sFlt-1 in a biological sample as described herein, wherein an increased concentration of sFlt-1 in the sample relative to a reference value for sFlt-1 concentration is indicative of heart failure, increased risk of heart failure, renal failure, or increased risk of renal failure.

The sFlt-1 concentration is deemed increased in comparison to a reference value, i.e. the sFlt-1 reference value as described herein. For example, an sFlt-1 plasma concentration useful as a reference value is about 308 pg/ml, but can be higher or about 380 pg/ml in plasma. The sFlt-1 concentration may be deemed increased as compared to the reference value when it is significantly higher, e.g. at least 20% higher (1.2 fold), more preferably at least 30% (1.3 fold) higher, even more preferably at least 40% higher (1.4 fold) or yet still more preferably 100% higher (2.0 fold).

To determine the concentration of sFlt-1, or of any other biomarker in the sample, any bioassay can be used that determines the concentration of the specific, targeted biomarker. For example, one or more specific binding agents can be used to analyze and determine the presence or absence of biomarkers in a sample. The biological sample is contacted with one or more agents that bind to the biomarker to determine the concentration or level of expression of the biomarker in the sample. One or more of the agents may be also operably linked to a detectable label. Immunoassay methods are suitable in this regard and may be carried out in any of a wide variety of formats. Immunological assay methods generally involve a reagent capable of specifically binding sFlt-1, and optionally a reagent capable of specific binding of an additional biomarker of heart failure. Suitable immunologic methods include, but are not limited to, immunoprecipitation, particle immunoassay, immunonephelometry, radioimmunoassay (RIA), enzyme immunoassay (EIA) including enzyme-linked immunosorbent assay (ELISA), sandwich, direct, indirect, or competitive ELISA assays, enzyme-linked immunospot assays (ELISPOT), fluorescent immunoassay (FIA), chemiluminescent immunoassay, flow cytometry assays, immunohistochemistry, Western blot, and protein-chip assays using for example antibodies, antibody fragments, receptors, ligands, or other agents binding the target analyte, such as sFlt-1. A general review of immunoassays is available in METHODS IN CELL BIOLOGY VOLUME 37: ANTIBODIES IN CELL BIOLOGY, Asai, ed. Academic Press, Inc. New York (1993), and BASIC AND CLINICAL IMMUNOLOGY 7^{TH} EDITION, Stites & Terr, eds. (1991), which are herein incorporated by reference in their entireties. For example, sFlt-1 and any other biomarker may be measured from any biological sample such as a blood plasma sample using the ARCHITECT™ immunoassay (Abbott Laboratories, Abbott Park, Ill.).

Generally, however, any other method that can detect or quantify biomarkers in a sample can be used in the methods. These methods include physical and molecular biology methods in addition to immunological methods. For example, suitable physical methods include mass spectrometric methods, fluorescence resonance energy transfer (FRET) assays, chromatographic assays, and dye-detection assays. Suitable molecular biology methods that can be used include, but are not limited to, Northern or Southern blot hybridization, nucleic acid dot- or slot-blot hybridization, in situ hybridization, nucleic acid chip assays, PCR, reverse transcriptase PCR (RT-PCR), or real time PCR (taq-man PCR). Other methods to detect biomarkers include, e.g., nuclear magnetic resonance (NMR), fluorometry, colorimetry, radiometry, luminometry, or other spectrometric methods, plasmon-resonance (e.g. BIACORE), and one- or two-dimensional gel electrophoresis.

Once measured, the concentration of sFlt-1 and that of any other additional biomarker being assessed is compared to a predetermined reference value for the specific biomarker. A measured, i.e. determined sFlt-1 concentration that exceeds the reference sFlt-1 value is indicative of heart failure or increased risk of heart failure in the subject. The reference value may be determined in one of several ways. For example, the sFlt-1 reference value can be the sFlt-1 concentration measured in a sample taken from a control subject, or may be the median sFlt-1 concentration calculated from the concentrations measured in multiple control samples taken from a group of control subjects. A median sFlt-1 concentration is preferably obtained from a group of at least 20 control subjects, more preferably at least 30, even more preferably at least 40.

A “control subject” is a healthy subject, i.e. a subject having no clinical signs or symptoms of a significant heart disease or heart failure. Preferably a control subject is clinically evaluated for otherwise undetected signs or symptoms of a significant heart disease or heart failure, which evaluation may include echocardiography and other routine laboratory testing.

Alternatively, an sFlt-1 cut-off value can be determined by a receiver operating curve (ROC) analysis from biological samples of a patient group. ROC analysis as generally well known in the biological arts is a determination of the ability of a test to discriminate one condition from another, e.g. diseased cases from normal cases, or to compare the diagnostic performance of two or more laboratory or diagnostic tests. A description of ROC analysis as applied according to the present disclosure is provided in P. J. Heagerty et al., Time-dependent ROC curves for censored survival data and a diagnostic marker, BIOMETRICS 56:337-44 (2000), the disclosure of which is hereby incorporated by reference in its entirety. Alternatively, an sFlt-1 cut-off value can be determined by a quartile analysis of biological samples of a patient group. For example, an sFlt-1 cut-off value can be determined by selecting a value that corresponds to any value in the 25th-75th percentile range, preferably a value that corresponds to the 25th percentile, the 50th percentile or the 75th percentile, and more preferably the 75th percentile. An exemplary sFlt-1 reference value obtained from the median of a relevant patient group is about 308 pg/ml in plasma. An exemplary sFlt-1 reference value obtained from quartile analysis at the 75th percentile is about 380 pg/ml in plasma.

The method may further include assessing at least one additional biomarker of heart failure, for example by measuring the concentration at least one additional biomarker in the biological sample, and comparing the measured concentration to a reference value for each additional biomarker being assessed. One, two, three, four or more additional biomarkers may be assessed. Additional such biomarkers of heart failure include but are not limited to B-type natriuretic peptide (BNP), NT-pro-BNP, pro-BNP, creatinine, PAPP-A, cardiac troponin I (TnI), cardiac troponin T (TnT), neuregulin-1, VEGF, PlGF, soluble CD40 ligand (sCD40L), myeloperoxidase (MPO), growth-differentiation factor 15 (GDF-15; see I. Anand et al., CIRCULATION. 122(14):1387-95 (2010)), soluble ST-2 protein (also known as IL1RL1; see B. Ky et al., CIRCULATION HEART FAILURE January 2011 e-publication ahead of print (doi: 10.1161/CIRCHEARTFAILURE.110.958223), copeptin (C-terminal provasopressin, CT-proAVP; see S. Masson et al., EUR J HEART FAIL 12, 338-347 (2010)), adrenomedullin (ADM; see S. von Haeling et al., EUR J HEART FAIL 12, 484-491 (2010)), high sensitivity C-reactive protein (hs-CRP), uric acid, and galectin-3 (gal-3; see D. Lok et al., CLIN RES CARDIOL 99:323-328 (2010)). A reference value may be similarly determined for any other biomarker of heart failure, as described herein with respect to determining a reference value for sFlt-1. Typically, a measured i.e., determined concentration of any additional biomarker in a biological sample that exceeds the reference value for that biomarker is also indicative of heart failure or increased risk of heart failure in the subject. Instances of biomarkers for which the opposite is true are nevertheless possible, i.e. biomarkers for which the relationship between concentration in a biological sample and instance of heart failure or increased risk of heart failure is inverse, such that a determined biomarker concentration that is below the reference value for the biomarker is indicative of heart failure or increased risk of heart failure in the subject.

For example, elevated levels of BNP or NT-pro-BNP in the blood have been used as diagnostic biomarkers of heart failure, and both BNP and NT-pro-BNP are approved biomarkers for acute congestive heart failure (CHF). BNP, also known as B-type natriuretic peptide or GC-B, is a 32 amino acid polypeptide expressed in the heart ventricles and secreted in response to excessive stretch of cardiac myocytes. NT-proBNP is a 76 amino acid N-terminal fragment that is co-secreted with BNP. Plasma concentrations of BNP and NT-pro-BNP are increased in patients with asymptomatic and symptomatic left ventricular dysfunction. Accordingly, the concentrations of both sFlt-1 and BNP may be determined and each compared to a corresponding predetermined reference value as described herein. A suitable reference value for BNP is, for example, a median of about 177 pg/ml in plasma. Similarly, the concentrations of both sFlt-1 and NT pro-BNP may be determined and each compared to a corresponding predetermined reference value as described herein. Neuregulin-1 has been described previously as a biomarker for prognosis or risk stratification of heart failure patients. (B. Ky et al., Neuregulin-1 beta is associated with disease severity and adverse outcomes in chronic heart failure, CIRCULATION 120:310-317 (2009)). For example, the concentrations of sFlt-1 and neuregulin-1, with or without one or more additional biomarkers such as BNP or NT pro-BNP may be determined, and each compared to a corresponding predetermined reference value as described herein.

With respect to a method for providing a diagnosis or prognosis of a subject having or at risk of having renal disease, the method may further include determining an estimated glomerular filtration rate (eGFR) in the subject, and comparing the determined eGFR with a reference eGFR value. eGFR can be readily determined from a blood sample according to well-known procedure. A determined concentration of eGFR greater than the reference eGFR value is further indicative of renal disease in the patient. The reference eGFR value may be may be that of a control as described herein, or may be the median eGFR calculated from the eGFR measured in multiple subjects comprising a group of control subjects. A median eGFR is preferably obtained from a group of at least 10 and preferably 20 control subjects, more preferably at least 30, even more preferably at least 40 control subjects. The method may further include assessing at least one additional biomarker of renal failure, for example by measuring the concentration at least one additional biomarker in the biological sample, and comparing the measured concentration to a reference value for each additional biomarker being assessed. Additional biomarkers of renal failure include but are not limited to serum creatinine, Cystatin C, NGAL, urinary interleukin 18, tubular enzymes such as the intestinal form of alkaline phosphatase, N-acetyl-beta-glucosaminidase and alanine-aminopeptidase, and kidney injury molecule 1 (KIM-1).

### C. KITS

The present disclosure also provides kits for assaying samples for presence and amount of sFlt-1 and optionally one or more additional biomarkers of heart failure, or one or more additional biomarker of renal failure. Such kits include one or more reagents useful for performing one or more immunoassays for detection and quantification of sFlt-1 and any one or more additional biomarkers. A kit generally includes a package with one or more containers holding the reagents, as one or more separate compositions or, optionally, as an admixture where the compatibility of the reagents will allow. The test kit can also include other material(s), which may be desirable from a user standpoint, such as a buffer(s), a diluent(s), a standard(s), and/or any other material useful in sample processing, washing, or conducting any other step of the assay.

A test kit may include for example an antibody specific for sFlt-1, and optionally one or more antibodies each specific for any additional biomarkers being used. Antibody reagents can be used as a positive control in immunoassays detecting the biomarkers. If desired, multiple concentrations of each antibody can be included in the kit to facilitate the generation of a standard curve to which the signal detected in the test sample can be compared. Alternatively, a standard curve can be generated by preparing dilutions of a single antibody solution provided in the kit.

Test kits according to the present disclosure may also include a solid phase, to which the antibodies functioning as capture antibodies and/or detection antibodies in a sandwich immunoassay format are bound. The solid phase may be a material such as a magnetic particle, a bead, a test tube, a microtiter plate, a cuvette, a membrane, a scaffolding molecule, a quartz crystal, a film, a filter paper, a disc or a chip. The kit may also include a detectable label that can be or is conjugated to an antibody, such as an antibody functioning as a detection antibody. The detectable label can for example be a direct label, which may be an enzyme, oligonucleotide, nanoparticle chemiluminophore, fluorophore, fluorescence quencher, chemiluminescence quencher, or biotin. Test kits may optionally include any additional reagents needed for detecting the label.

Test kits according to the present disclosure preferably include instructions for carrying out one or more immunoassays for detecting and quantifying biomarker concentration in a sample, including sFlt-1 concentration and concentration of any additional biomarkers of heart failure being used. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

It will be readily apparent to those skilled in the art that other suitable modifications and adaptations of the methods of the present disclosure described herein are obvious and may be made using suitable equivalents without departing from the scope of the present disclosure or the embodiments disclosed herein. Having now described the present disclosure in detail, the same will be more clearly understood by reference to the following example which is included for purposes of illustration only and not intended to limit the scope of the present disclosure. The disclosures of all journal references, U.S. patents and publications referred to herein are hereby incorporated by reference in their entireties.

### EXAMPLE

### Measurement of Biomarkers

Background: The Penn Heart Failure Study (PHFS) is a multi-center prospective cohort study of outpatients with primarily chronic systolic heart failure recruited from referral centers at the University of Pennsylvania (Philadelphia, Pa.), Case Western University (Cleveland, Ohio), and the University of Wisconsin (Madison, Wis.) (20, 21). The primary inclusion criterion was a clinical diagnosis of heart failure. Participants were excluded if they had a non-cardiac condition resulting in an expected mortality of less than 6 months as judged by the treating physician, or if they were unable or unwilling to provide informed consent. At time of study entry, detailed clinical data were obtained using a standardized questionnaire administered to the patient and treating physician, with verification via medical records. Venous blood samples were obtained at enrollment, processed, and stored at −80° C. until time of assay. Follow-up events including all-cause mortality and cardiac transplantation were prospectively ascertained every 6 months via direct patient contact and verified through death certificates, medical records, and contact with patients' family members by dedicated research personnel. All participants provided written, informed consent, and the PHFS protocol was approved by participating Institutional Review Boards.

Biomarkers Assays: All biomarkers were measured from the same aliquot from a banked plasma sample that was obtained at time of study entry. PlGF (placental growth factor) and sFlt-1 (soluble Fms-like tyrosine kinase receptor 1) were measured using prototype ARCHITECT™ immunoassays (Abbott Laboratories, Abbott Park, Ill.). The sFlt-1 immunoassay measures both free and bound sFlt-1. The assay range was 15 to 50,000 pg/ml. The intra- and interassay Coefficients of variation (CV) ranged from 1.3% to 5.2% and 1.9% to 5.9%, respectively. The PlGF immunoassay measures the free, not bound PlGF-1 with approximately 20% cross-reactivity with the PlGF-2 isoform. The assay range was 1 to 1,500 pg/mL. The intra- and interassay CV ranged from 1.4% to 6.7% and 1.8% to 6.7%, respectively.

BNP (B-type natriuretic peptide) was measured using the ARCHITECT™ BNP chemiluminescent microparticle immunoassay (Abbott Laboratories, Abbott Park, Ill.) as previously described (22). The assay range was 10 to 5,000 pg/ml. The intra- and interassay CV ranged from 0.9% to 5.6% and 1.7% to 6.7%, respectively.

Statistical Methods: Baseline characteristics were summarized for the entire cohort using standard descriptive statistics. Independent determinants of baseline sFlt-1 and PlGF were ascertained using a multivariable linear regression model for each marker. The inclusion of adjustment variables was defined based on clinical judgment and statistical significance according to a stepwise model-selection procedure based on Akaike information criteria (AIC).

Cox regression models were used to determine the unadjusted association between sFlt-1 and PlGF and time to the combined outcome of all-cause death or cardiac transplantation. Biomarkers were modeled both as continuous variables and using quartiles. Adjusted models included covariates based on clinical judgment and statistical evidence for confounding as determined above. In addition, heart failure severity was adjusted for by stratifying the baseline hazard function by the New York Heart Association (NYHA) functional class. Differences in sFlt-1 and PlGF associations across groups defined by cardiomyopathy etiology (i.e., ischemic versus nonischemic) and estimated Glomerular Filtration Rate (eGFR; dichotomized at the median) were evaluated using interaction terms with the continuous form of the biomarker variable.

The joint effects of sFlt-1 and BNP were evaluated by dividing the cohort into groups based on the median level of each marker. In addition, time-dependent receiver operating characteristic (ROC) curves were used to compare the ability of markers to classify patients with regard to death or cardiac transplantation at 1 year (23). Confidence intervals for the area under the ROC curve (AUC) were obtained from 1,000 bootstrapped samples, and AUCs were compared using Wald tests. All statistical analyses were completed using R 2.11.0, including the MASS, survival, and survivalROC packages (24-27).

Baseline Characteristics: Biomarker data were available in 1,535 subjects. Twenty-four subjects whose sFlt-1 or PlGF was greater than the 99th percentile were excluded from the analysis given that the levels in these patients were most reflective of the influence of other disease states not related to heart failure (e.g. pregnancy, infection, inflammation, or cancer). Complete data on baseline characteristics and outcomes were available on 1,412 (92%) subjects.

The clinical characteristics of the 1,412 patients with complete data are shown in Table 1. The majority of the patients were male (67%) and Caucasian (74%), with a mean age across the cohort of 56 years. There were 426 patients (30%) with an ischemic cause of heart failure, 398 (28%) patients with a history of diabetes and 821 (58%) with a history of hypertension.

TABLE 1
Baseline Characteristics

| | Entire Cohort |
|---|---|
| | n = 1412 |
| Demographic Characteristics | |
| Age, years | 56 (14) |
| Male, n (%) | 947 (67%) |
| Race, n (%) | |
| Caucasian | 1041 (74%) |
| African American | 321 (23%) |
| Other | 50 (3%) |
| Medical History and Risk Factors | |
| History of hypertension, n (%) | 821 (58%) |
| History of diabetes, n (%) | 398 (28%) |
| Any peripheral vascular disease, n (%) | 185 (13%) |
| Tobacco use, n (%) | |
| Never | 520 (37%) |
| Current | 129 (9%) |
| Former | 763 (54%) |
| Heart Failure Characteristics | |
| NYHA functional classification, n (%) | |
| I | 229 (16%) |
| II | 651 (46%) |
| III | 422 (30%) |
| IV | 110 (8%) |
| Ischemic heart failure, n (%) | 426 (30%) |
| Ejection fraction, % | 33.1 (17) |
| Cardiac resynchronization therapy, n (%) | 356 (25%) |
| Medication Use | |
| ACE inhibitors or angiotensin receptor blockers, n (%) | 1231 (87%) |
| Beta blockers, n (%) | 1230 (87%) |
| Aspirin, n (%) | 700 (50%) |
| HMG CoA reductase inhibitors, n (%) | 767 (54%) |
| Clinical Measures | |
| Body mass index, kg/m² | 30.0 (7.2) |
| Systolic blood pressure | 114 (21) |
| Pulse pressure, mmHg | 45 (15) |
| Estimated Glomerular Filtration Rate (eGFR), | 68.5 (26) |
| ml/min/1.73 m² | |
| BNP, pg/ml, median (IQR) | 177 (48.7, 584) |
| sFlt-1, pg/ml | 350 (190) |
| PlGF, pg/ml | 19.5 (6.2) |

Summaries presented as mean (standard deviation) unless otherwise noted as n (%) or median (inter-quartile range; IQR)

Clinical Factors Associated with Baseline sFlt-1 and PlGF Levels: Across the cohort, the distributions of sFlt-1 and PlGF were approximately normal, with slightly heavier positive tails than would be expected if their distributions were truly normal. Multivariable models were used to determine clinical factors that independently influenced baseline levels of each biomarker. sFlt-1 levels ranged from 115 to 2012 pg/ml, with a mean±s.d. of 350±190 pg/ml. As shown in Table 2, African American race, higher NYHA class, and higher plasma BNP were each associated with higher levels of sFlt-1. Increasing age, aspirin use, higher estimated glomerular filtration rate (eGFR) and higher pulse pressure were each associated with lower levels of sFlt-1. For PlGF, levels ranged from 0.7 to 42.3 pg/ml, with a mean of 19.5±6.2 pg/ml.

TABLE 2
Independent Determinants of Baseline sFlt-1 Levels

| | Difference | | |
|---|---|---|---|
| | in sFlt1* | 95% CI | p value |
| Demographic Characteristics | | | |
| Age (10 year difference) | −7.9 | (−15, −0.66) | 0.02 |
| African American (vs Caucasian) | +70 | (49, 91) | <0.01 |
| Heart Failure Characteristics | | | |
| NYHA functional classification | | | |
| II (vs I) | +7.2 | (−17, 32) | 0.56 |
| III (vs I) | +53 | (26, 80) | <0.01 |
| IV (vs I) | +220 | (180, 250) | <0.01 |
| Medical History and Risk Factors | | | |
| Hypercholesterolemia (vs none) | +21 | (1.0, 40) | 0.04 |
| Medication Use | | | |
| Beta-blockers (vs none) | −30 | (−55, −4.6) | 0.02 |
| Aspirin (vs none) | −24 | (−43, −6.3) | 0.01 |
| Clinical Measures | | | |
| eGFR (10 ml/min/1.73 m2 increase) | −5.0 | (−8.8, −1.2) | <0.01 |
| Sodium (per 1-unit increase) | −5.1 | (−7.7, 2.6) | <0.01 |
| BNP (multiplicative difference of 2) | +17 | (13, 21) | <0.01 |

*This column denotes the β coefficient from a multivariable linear regression model for sFlt-1, and represents the difference in mean sFlt-1 (pg/ml) between each group for categorical or continuous variables. The mean ± SD of sFlt-1 was 348 ± 181 pg/ml.

As shown in Table 3 below, increasing age, male gender, history of hypertension, diabetes, peripheral vascular disease, higher pulse pressure, use of cardiac resynchronization, and higher BNP were each associated with higher PlGF levels. African American race, angiotensin converting enzyme (ACE) inhibitor or angiotensin receptor blocker (ARB) use and higher eGFR were each associated with lower levels of PlGF.

TABLE 3
Independent Determinants of Baseline PlGF Levels

| | Difference | | |
|---|---|---|---|
| | in PlGF* | 95% CI | p value |
| Demographic Characteristics | | | |
| Age (10 year difference) | +0.57 | (0.31, 0.84) | <0.01 |
| Male (vs female) | +0.98 | (0.34, 1.6) | <0.01 |
| African American (vs Caucasian) | −3.0 | (−3.8, −2.3) | <0.01 |
| Medical History and Risk Factors | | | |
| History of diabetes (vs none) | +1.1 | (0.40, 1.8) | <0.01 |
| Heart Failure Characteristics | | | |
| Cardiac resynchronization | +0.88 | (0.17, 1.6) | 0.02 |
| therapy (vs none) | | | |
| Medication Use | | | |
| ACE inhibitors or ARBs (vs none) | −1.1 | (−2.0, −0.21) | 0.02 |
| Clinical Measures | | | |
| Pulse pressure (10 mmHg difference) | +0.43 | (0.23, 0.64) | <0.01 |
| eGFR (10 ml/min/1.73 m2 difference) | −0.26 | (−0.39, −0.11) | <0.01 |
| BNP (multiplicative difference of 2) | +0.21 | (0.065, 0.35) | <0.01 |

*This column denotes the β coefficient from a multivariable linear regression model for PlGF, and represents the difference in mean PlGF (pg/ml) between each group for categorical or continuous variables. The mean ± SD of PlGF was 19.4 ± 6.2 pg/ml

Comparing Tables 2 and 3, sFlt-1 levels appear to be influenced by fewer factors than PlGF. However, sFlt-1 correlated more specifically with heart failure measures than PlGF, as sFlt-1 was independently associated with two measures of heart failure severity (NYHA Class and BNP) whereas PlGF was only weakly associated with BNP.

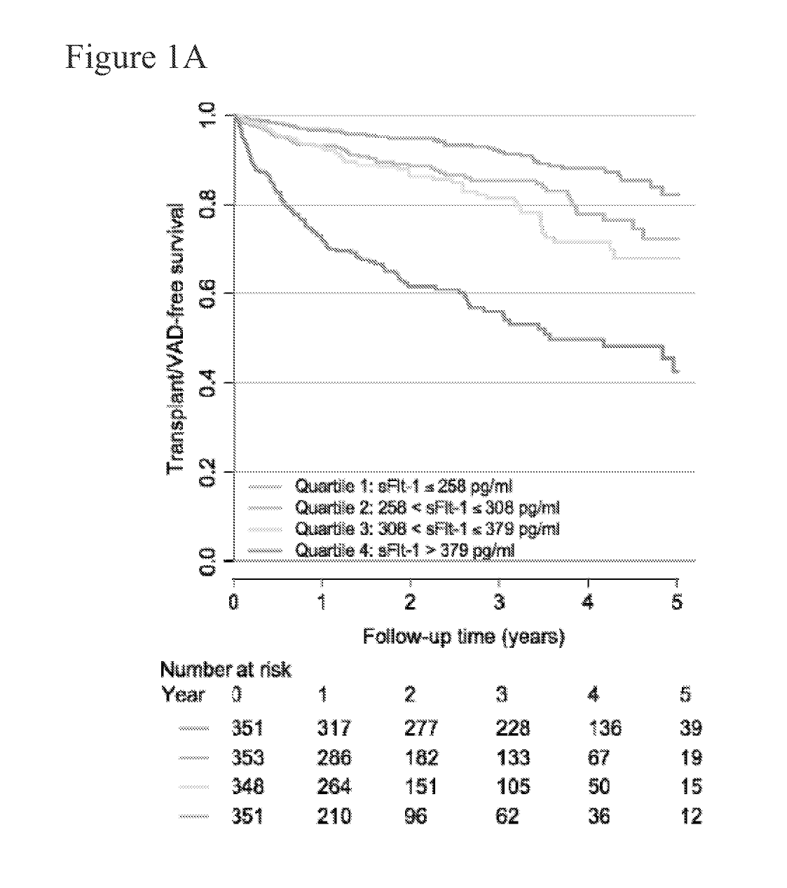

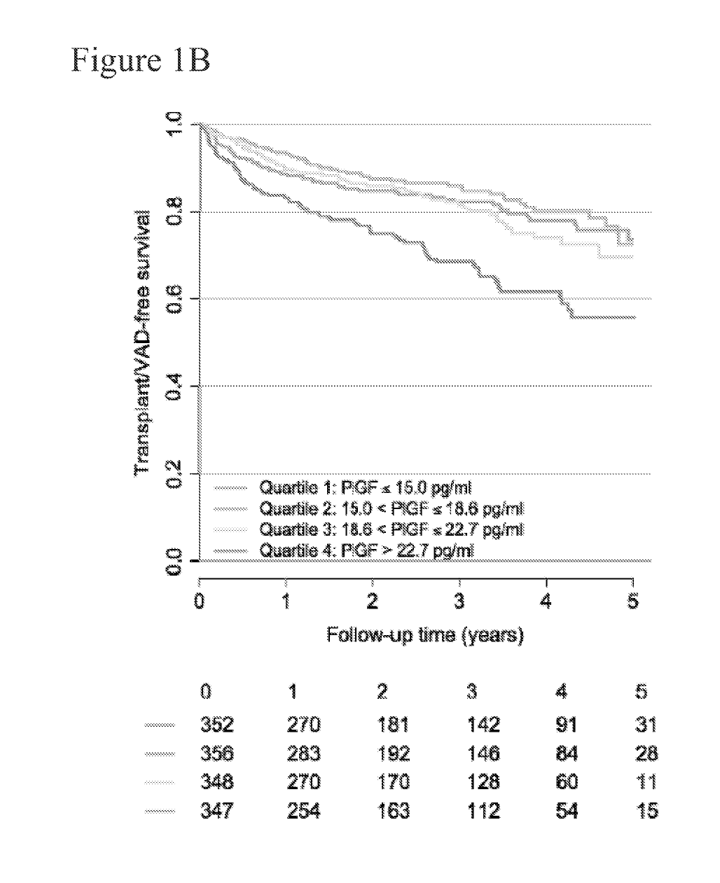

Associations between sFlt-1 and PlGF and Adverse Clinical Outcomes: Over a median follow-up time of 2 years, 175 deaths and 103 transplants occurred, and 27 LVADs were implanted. FIG. 1 illustrates transplant-free survival according to baseline levels of sFlt-1 (A) and PlGF (B). In particular, Kaplan-Meier plots were used to illustrate the incidence of all-cause death, cardiac transplantation or ventricular assist device (VAD) placement among Penn Heart Failure Study participants according to baseline sFlt-1 (A) and PlGF (B) Levels (P<0.01 by log rank test for each panel).

In unadjusted models comparing the 4th versus 1st quartile, those patients with a circulating sFlt-1 level>379 pg/ml had a 6.17-fold increased risk of adverse outcomes (p<0.01) (see Table 4 below and FIG. 1A).

TABLE 4
Association of sFLT-1 and PlGF with Risk of All-Cause Death Cardiac
Transplantation or VAD Placement

| | sFlt-1 | | PlGF | |
|---|---|---|---|---|
| | HR (95% CI) | p value | HR (95% CI) | p value |
| Model 1 | | | | |
| Quartile 1 | Referent | | Referent | |
| Quartile 2 | 1.76 (1.16, 2.66) | 0.01 | 0.83 (0.58, 1.19) | 0.31 |
| Quartile 3 | 2.21 (1.47, 3.31) | <0.01 | 1.06 (0.75, 1.50) | 0.76 |
| Quartile 4 | 6.17 (4.30, 8.86) | <0.01 | 1.89 (1.39, 2.58) | <0.01 |
| Per SD increase | 1.46 (1.37, 1.54) | <0.01 | 1.31 (1.18, 1.46) | <0.01 |
| Model 2 | | | | |
| Quartile 1 | Referent | | Referent | |
| Quartile 2 | 1.42 (0.93, 2.19) | 0.10 | 0.84 (0.58, 1.23) | 0.38 |
| Quartile 3 | 1.53 (1.00, 2.33) | 0.05 | 0.93 (0.64, 1.35) | 0.71 |
| Quartile 4 | 2.61 (1.72, 3.96) | <0.01 | 1.39 (0.98, 1.97) | 0.07 |
| Per SD increase | 1.19 (1.10, 1.28) | <0.01 | 1.10 (0.98, 1.23) | 0.10 |
| Model 3 | | | | |
| Quartile 1 | Referent | | Referent | |
| Quartile 2 | 1.24 (0.81, 1.90) | 0.33 | 0.93 (0.64, 1.36) | 0.72 |
| Quartile 3 | 1.16 (0.75, 1.80) | 0.50 | 0.94 (0.65, 1.37) | 0.76 |
| Quartile 4 | 1.67 (1.06, 2.63) | 0.03 | 1.36 (0.96, 1.93) | 0.08 |
| Per SD increase | 1.14 (1.05, 1.24) | <0.01 | 1.11 (0.99, 1.25) | 0.05 |
| Model 4 | | | | |
| Quartile 1 | Referent | | Referent | |
| Quartile 2 | 1.40 (0.93, 2.13) | 0.11 | 0.72 (0.50, 1.03) | 0.07 |
| Quartile 3 | 1.36 (0.90, 2.07) | 0.14 | 0.76 (0.53, 1.08) | 0.12 |
| Quartile 4 | 2.54 (1.76, 2.27) | <0.01 | 1.04 (0.75, 1.42) | 0.83 |
| Per SD increase | 1.26 (1.17, 1.36) | <0.01 | 1.05 (0.94, 1.17) | 0.44 |

HR = hazard ratio;
CI = confidence interval;
SD = standard deviation
Model 1: Unadjusted
Model 2: Adjusted for age, gender, race, NYHA functional classification, history of diabetes, tobacco use, ischemic cardiomyopathy etiology, internal cardiac defibrillator, cardiac resynchronization therapy, ACE inhibitor/angiotensin receptor blocker use, aldosterone use, beta blocker use, HMG CoA reductase inhibitor use, body mass index, and clinical site
Model 3: Adjusted for Model 2 covariates and log₂-transformed BNP
Model 4: Adjusted for SHFM score

After adjustment for demographics, heart failure characteristics, and clinical measures including BNP, this association was attenuated in magnitude but remained statistically significant (HR 1.67, 95% CI 1.06-2.63, p=0.03 comparing 4th quartile to 1st quartile). After adjustment for established clinical risk scores such as the Seattle Heart Failure Model, this association was attenuated to a lesser degree (HR 2.54, 95% CI 1.76-2.27, p<0.01). Similar results were obtained when sFlt-1 was modeled as a continuous variable and with various percentile cutpoints. In contrast, patients in the highest quartile of PlGF (>22.7 pg/ml) had only a 1.89 fold increased risk which did not remain significant in adjusted models (see Table 4 and FIG. 1B). As in the cross-sectional analyses, these findings support a role for sFlt-1 as an independent biomarker of heart failure severity, whereas PlGF had no independent associations with outcomes. Associations between vascular growth factors and outcomes might differ based upon the disease states which influence the biology of these markers, including the underlying etiology of heart failure and renal dysfunction (11, 19).

To explore these possibilities, secondary analyses were performed that included interaction terms between biomarker levels (modeled continuously) and heart failure etiology (ischemic or nonischemic) and between biomarker levels and renal function (above or below median eGFR). In contrast to previously published reports suggesting greater relevance of PlGF and sFlt-1 in ischemic heart disease, there was no significant interaction by heart failure etiology on the association between either marker and outcome (interaction p=0.18 for sFlt-1; p=0.41 for PlGF). Interaction by renal function was also not observed in connection with the relationship between PlGF and outcomes. However, there was a significant interaction between eGFR and sFlt-1 and the association of adverse outcomes (p<0.01). In those patients with an eGFR less than the median of 67.8 ml/min/1.73 m2, associations between sFlt-1 and outcomes were attenuated (HR 1.00, 95% CI 0.90-1.12, p=0.95). By contrast, in patients with an eGFR greater than the median, the adjusted risk of adverse outcomes with each standard deviation increase in sFlt-1 was 1.30 (95% CI 1.14-1.47, p<0.01), indicating that assessment of sFlt-1 may be more useful in the setting of normal renal function.

Combined use of sFlt-1 and BNP: The utility of the joint assessment of sFlt-1 and the clinically used biomarker BNP was explored in predicting adverse outcomes. There was a moderate correlation between levels of sFlt-1 and BNP (R=0.54, p<0.01), and their combined use was important in risk assessment (see Table 5 below, and FIG. 1).

TABLE 5
Joint Effects of sFlt-1 and BNP on Risk of All-Cause Death
or Cardiac Transplantation or VAD Placement

| | | | Unadjusted | | Adjusted** | |
|---|---|---|---|---|---|---|
| | | | | p | HR | p |
| sFlt-1* | BNP* | n | HR (95% CI) | value | (95% CI) | value |
| Low | Low | 482 | Referent | | Referent | |
| High | Low | 219 | 1.44 (0.86, 2.40) | 0.16 | 1.21 (0.71, | 0.48 |
| | | | | | 2.14) | |
| Low | High | 219 | 2.78 (1.84, 4.19) | <0.01 | 1.99 (1.30, | <0.01 |
| | | | | | 3.05) | |
| High | High | 483 | 6.08 (4.36, 8.50) | <0.01 | 2.87 (1.96, | <0.01 |
| | | | | | 4.21) | |

HR = hazard ratio;
CI = confidence interval
*Low/high sFlt-1 defined as below/above median (308 pg/ml)
*Low/high BNP defined as below/above median (175 pg/ml)
**Adjusted for all covariates listed in Table 4, Model 2

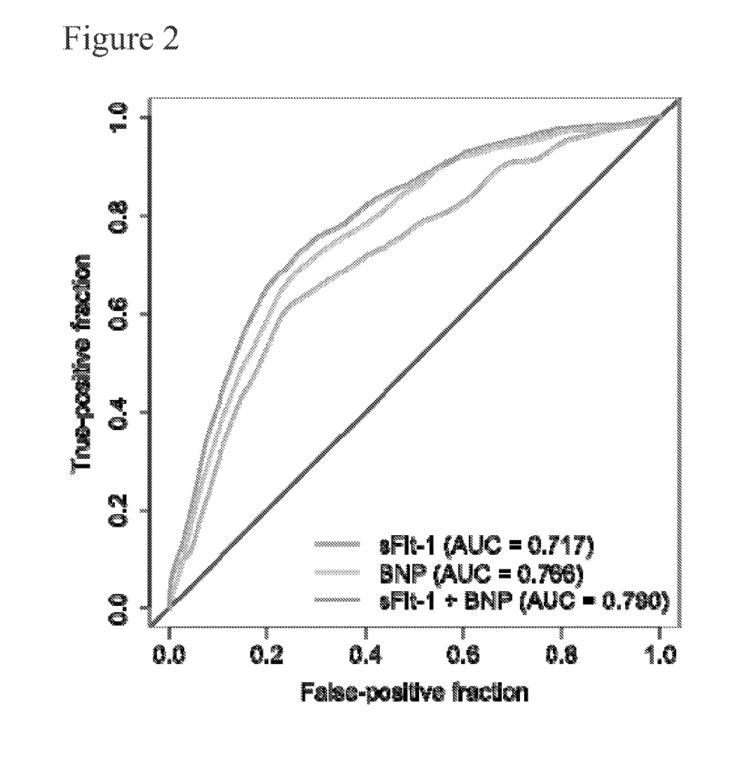

Compared to the referent group of patients with levels of both markers less than the median, patients with elevations in both sFlt-1 and BNP had a markedly elevated risk estimate than either marker alone, and this association remained significant in multivariable adjusted models (HR 2.87, 95% CI 1.96-4.21, p<0.01). Furthermore, in the group of patients with high BNP levels, the combination of a high sFlt-1 level was associated with a 1.5 to 2 fold increase in risk compared to those patients with low sFlt-1 levels (p<0.01 unadjusted, p=0.04 adjusted). FIG. 2 illustrates ROC curves for baseline sFlt-1 and BNP at 1 Year. In particular, the ROC curves compare the ability of baseline sFlt-1 and BNP to correctly classify patients who died, required heart transplantation, or VAD placement at 1-year of follow-up (p<0.05 for AUC comparing sFlt-1 and BNP versus BNP alone or versus sFlt-1 alone). As shown in FIG. 2, in ROC analysis at 1 year, sFlt-1 and BNP in combination (AUC=0.79) showed greater accuracy in identifying patients who died, required heart transplantation or VAD placement than did sFlt-1 alone (AUC=0.72, p<0.05) or BNP alone (AUC=0.77, p<0.05). These findings demonstrate an improved ability to discern high and low risk heart failure patients using both sFlt-1 and BNP compared to the current standard of assessing BNP alone.

### REFERENCES

1. Oka T, Komuro I. Molecular mechanisms underlying the transition of cardiac hypertrophy to heart failure. Circ J. 2008; 72 Suppl A:A13-6.
2. Lip G Y, Chung I. Vascular endothelial growth factor and angiogenesis in heart failure. J Card Fail. 2005 May; 11:285-7.
3. Hsieh P C, Davis M E, Lisowski L K, Lee R T. Endothelial-cardiomyocyte interactions in cardiac development and repair. Annu Rev Physiol. 2006; 68:51-66.
4. Helotera H, Alitalo K. The VEGF family, the inside story. Cell. 2007 Aug. 24; 130:591-2.
5. Carmeliet P. Angiogenesis in life, disease and medicine. Nature. 2005 Dec. 15; 438:932-6.
6. Abraham D, Hofbauer R, Schafer R, et al. Selective downregulation of VEGF-A(165), VEGFR(1), and decreased capillary density in patients with dilative but not ischemic cardiomyopathy. Circ Res. 2000 Oct. 13; 87:644-7.
7. Izumiya Y, Shiojima I, Sato K, Sawyer D B, Colucci W S, Walsh K. Vascular endothelial growth factor blockade promotes the transition from compensatory cardiac hypertrophy to failure in response to pressure overload. Hypertension. 2006 May; 47:887-93.
8. Pepe M, Mamdani M, Zentilin L, et al. Intramyocardial VEGF-B 167 gene delivery delays the progression towards congestive failure in dogs with pacing-induced dilated cardiomyopathy. Circ Res. 2010 Jun. 25; 106:1893-903.
9. Zhao Q, Ishibashi M, Hiasa K, Tan C, Takeshita A, Egashira K. Essential role of vascular endothelial growth factor in angiotensin II-induced vascular inflammation and remodeling. Hypertension. 2004 September; 44:264-70.
10. Luttun A, Tjwa M, Carmeliet P. Placental growth factor (PlGF) and its receptor flt-1 (VEGFR-1): Novel therapeutic targets for angiogenic disorders. Ann N Y Acad Sci. 2002 December; 979:80-93.
11. Onoue K, Uemura S, Takeda Y, et al. Usefulness of soluble fms-like tyrosine kinase-1 as a biomarker of acute severe heart failure in patients with acute myocardial infarction. Am J. Cardiol. 2009 Dec. 1; 104:1478-83.
12. Heeschen C, Dimmeler S, Fichtlscherer S, et al. Prognostic value of placental growth factor in patients with acute chest pain. JAMA. 2004 Jan. 28; 291:435-41.
13. Levine R J, Maynard S E, Qian C, et al. Circulating angiogenic factors and the risk of preeclampsia. N Engl J. Med. 2004 Feb. 12; 350:672-83.
14. Noori M, Donald A E, Angelakopoulou A, Hingorani A D, Williams D J. Prospective study of placental angiogenic factors and maternal vascular function before and after preeclampsia and gestational hypertension. Circulation. 2010 Jul. 19.
15. Iwama H, Uemura S, Naya N, et al. Cardiac expression of placental growth factor predicts the improvement of chronic phase left ventricular function in patients with acute myocardial infarction. J Am Coll Cardiol. 2006 Apr. 18; 47:1559-67.
16. Lenderink T, Heeschen C, Fichtlscherer S, et al. Elevated placental growth factor levels are associated with adverse outcomes at four-year follow-up in patients with acute coronary syndromes. J Am Coll Cardiol. 2006 Jan. 17; 47:307-11.
17. Belgore F M, Blann A D, Lip G Y. sFlt-1, a potential antagonist for exogenous VEGF. Circulation. 2000 Oct. 10; 102:E108-9.
18. Kodama Y, Kitta Y, Nakamura T, et al. Atorvastatin increases plasma soluble fms-like tyrosine kinase-1 and decreases vascular endothelial growth factor and placental growth factor in association with improvement of ventricular function in acute myocardial infarction. J Am Coll Cardiol. 2006 Jul. 4; 48:43-50.
19. Nakamura T, Funayama H, Kubo N, et al. Elevation of plasma placental growth factor in the patients with ischemic cardiomyopathy. Int J. Cardiol. 2009 Jan. 9; 131:186-91.
20. Vorovich E, Chuai S, Li M, et al. Comparison of MMP-9 and BNP as clinical biomarkers in chronic heart failure. American Heart Journal. 2008; 155: 992-997.
21. Ky B, Kimmel S E, Safa R N, et al. Neuregulin-1 beta is associated with disease severity and adverse outcomes in chronic heart failure. Circulation. 2009; 120:310-317.
22. Yeo K T, Wu A H, Apple F S, et al. Multicenter evaluation of the roche NT-proBNP assay and comparison to the biosite triage BNP assay. Clin Chim Acta. 2003 December; 338:107-15.
23. Heagerty P J, Lumley T, Pepe M S. Time-dependent ROC curves for censored survival data and a diagnostic marker. Biometrics. 2000 June; 56:337-44.
24. Heagerty P J, Saha P. SurvivalROC: Time-dependent ROC curve estimation from censored survival data. 2006;R package version 1.0.0.
25. R Development Core Team. R; A language and environment for statistical computing. 2009.
26. Therneau T, Lumley T. Survival: Survival analysis, including penalized likelihood. 2009;R package version 2.35-4.
27. Venables W N, Risch N J. Modern applied statistics with S. 4th ed. New York: Springer, 2002.
28. Tammela T, Enholm B, Alitalo K, Paavonen K. The biology of vascular endothelial growth factors. Cardiovasc Res. 2005 Feb. 15; 65:550-63.
29. Maynard S E, Min J Y, Merchan J, et al. Excess placental soluble fms-like tyrosine kinase 1 (sFlt1) may contribute to endothelial dysfunction, hypertension, and proteinuria in preeclampsia. J Clin Invest. 2003 March; 111:649-58.
30. Belgore F M, Blann A D, Lip G Y. sFlt-1, a potential antagonist for exogenous VEGF. Circulation. 2000 Oct. 10; 102:E108-9.
31. Willett C G, Duda D G, di Tomaso E, et al. Efficacy, safety, and biomarkers of neoadjuvant bevacizumab, radiation therapy, and fluorouracil in rectal cancer: A multidisciplinary phase II study. J Clin Oncol. 2009 Jun. 20; 27:3020-6.
32. Di Marco G S, Reuter S, Hillebrand U, et al. The soluble VEGF receptor sFlt1 contributes to endothelial dysfunction in CKD. J Am Soc Nephrol. 2009 October; 20:2235-45.

What is claimed is:

1. A method for providing a diagnosis, prognosis or risk classification of a subject having or at risk of having heart failure, the method comprising:
a) providing a biological sample from the subject;
b) determining the concentration of soluble Flt-1 (sFlt-1) in the sample; and
c) comparing the determined sFlt-1 concentration with a reference sFlt-1 value, wherein a determined sFlt-1 concentration of the subject greater than the reference sFlt-1 value is indicative of heart failure or increased risk of heart failure in the subject.

2. The method of claim 1, further comprising assessing at least one additional biomarker of heart failure.

3. The method according to claim 1 or 2, wherein the sFlt-1 reference value is the sFlt-1 concentration of a control sample or a sFlt-1 cut-off value.

4. The method according to any one of claim 1 or 2, wherein the sFlt-1 concentration is the sFlt-1 plasma concentration.

5. The method according to claim 3, wherein the control sample is selected from a biological sample of a control subject and an sFlt-1 standard.

6. The method according to claim 3, wherein the sFlt-1 concentration of a control sample is the median sFlt-1 concentration of a plurality of control samples from a group of control subjects.

7. The method according to claim 3, wherein the sFlt-1 cut-off value is determined by a receiver operating curve (ROC) analysis from biological samples of a patient group.

8. The method according to claim 3, wherein the sFlt-1 cut-off value is determined by a quartile analysis of biological samples of a patient group.

9. The method according to claim 3, wherein the sFlt-1 cut-off value is about 308 pg/ml in plasma.

10. The method according to claim 1, wherein providing a diagnosis is providing a diagnosis of heart failure.

11. The method according to claim 1, wherein providing a prognosis is selected from determining heart failure severity and risk assessment of the subject with heart failure.

12. The method according to claim 1, wherein heart failure is selected from the group consisting of: chronic heart failure, systolic heart failure, dilated cardiomyopathy (DCM), ischemic cardiomyopathy, acute myocardial infarction, left ventricular dysfunction, and right ventricular dysfunction.

13. The method according to claim 1, further comprising the assessment of at least one additional biomarker of heart failure, wherein the additional biomarker of heart failure is selected from the group consisting of: B-type natriuretic peptide (BNP), NT-pro-BNP, pro-BNP, creatinine, PAPP-A, cardiac troponin I (TnI), cardiac troponin T (TnT), neuregulin-1, VEGF, PlGF, soluble CD40 ligand (sCD40L), myeloperoxidase (MPO), growth-differentiation factor 15 (GDF-15), soluble ST-2 protein, copeptin, adrenomedullin, high sensitivity C-reactive protein (hs-CRP), uric acid, and galectin-3 (gal-3).

14. The method according to claim 13, wherein the assessment of at least one additional biomarker for heart failure comprises measuring the concentration of the at least one additional biomarker in a biological sample of the subject.

15. The method according to claim 14, further comprising comparing the measured concentration of the at least one additional biomarker with a reference value.

16. The method according to claim 15, wherein the additional biomarker is BNP and the reference value for BNP is a cut-off value of about 177 pg/ml in plasma.

17. The method according to claim 15, wherein the reference value is the biomarker concentration of a control sample or a biomarker cut-off value.

18. The method according to claim 1, wherein the biological sample of the subject and/or the control sample is taken from a human.

19. The method according to claim 18, wherein the sample is selected from a bodily fluid, whole blood, plasma, serum, urine and cell culture suspensions or fractions thereof.

20. The method according to claim 18, wherein the sample is blood plasma or blood serum.

21. The method according to claim 20, wherein a coagulation inhibitor is added to peripheral blood.

22. The method according to claim 1, wherein determining the concentration of sFlt-1 and the at least one further biomarker is carried out by using an immunological method and molecules binding to sFlt-1 and the biomarker.

23. A method for identifying patients or patient subgroups having an increased cardiac risk, the method comprising:
a) providing a biological sample from at least one patient having or suspected of having an increased cardiac risk compared to a reference cardiac risk;
b) determining the concentration of soluble Flt-1 (sFlt-1) in the sample, and
c) comparing the determined sFlt-1 concentration with at least one reference value, wherein a determined concentration of sFlt-1 greater than the reference value is indicative of increased cardiac risk of the patient.

24. The method according to claim 23, further comprising assessing at least one additional biomarker of increased cardiac risk.

25. The method according to claim 23, wherein the reference value is the sFlt-1 concentration of a control sample or an sFlt-1 cut-off value.

26. The method according to claim 25, wherein the sFlt-1 concentration is the sFlt-1 plasma concentration.

27. The method according to claim 25, wherein the control sample is selected from a biological sample of a control subject and an sFlt-1 standard.

28. The method according to any one of claims 25-27, wherein the sFlt-1 concentration of a control sample is the median sFlt-1 concentration of control samples of a group of control subjects.

29. The method according to claim 25, wherein the sFlt-1 cut-off value is determined by a receiver operating curve (ROC) analysis from biological samples of a patient group.

30. The method according to claim 25, wherein the sFlt-1 cut-off value is determined by a quartile analysis of biological samples of a patient group.

31. The method according to claim 30, wherein the sFlt-1 cut-off value is about 308 pg/ml in plasma.

32. A method for diagnosis, prognosis and/or risk stratification of cardiovascular disease in a subject having or suspected of having heart failure, the method comprising the detection of an increased sFlt-1 concentration in the subject.

33. A method for providing a diagnosis or prognosis of a subject having or at risk of having renal disease, the method comprising:
a) providing a biological sample from at least one patient having or at risk of having renal disease;
b) determining the concentration of soluble Flt-1 (sFlt-1) in the sample, and
c) comparing the determined sFlt-1 concentration with at least one reference value, wherein a determined concentration of sFlt-1 greater than the reference value is indicative of renal disease in the patient.

34. The method of claim 33, further comprising determining an estimated glomerular filtration rate (eGFR) in the subject, comparing the determined eGFR with a reference eGFR value, wherein a determined concentration of eGFR greater than the reference eGFR value is further indicative of renal disease in the patient.

35. The method according to claim 33 or 34, wherein the reference value is the sFlt-1 concentration of a control sample or an sFlt-1 cut-off value.

36. The method according to claim 33 or 34, wherein the sFlt-1 concentration is the sFlt-1 plasma concentration.

37. The method according to claim 33 or 34, wherein the control sample is selected from a biological sample of a control subject and an sFlt-1 standard.

38. The method according to claim 33 or 34, wherein the sFlt-1 concentration of a control sample is the median sFlt-1 concentration of control samples of a group of control subjects.

39. The method according to claim 33 or 34, wherein the reference eGFR value is the median eGFR of a group of control subjects.

40. A kit for performing a method according to any one of claim 1, 23 or 33, the kit comprising:
a) at least one reagent capable of specifically binding sFlt-1 to quantify the sFlt-1 concentration in a biological sample of a subject, and
b) a reference standard indicating a reference sFlt-1 concentration.

41. The kit according to claim 40 for performing a method for providing a diagnosis, prognosis or risk classification of one or more subjects having or at risk of having heart failure, further comprising at least one additional reagent capable of specifically binding at least one additional biomarker of heart failure in the biological sample to quantify the concentration of the at least one additional biomarker in the biological sample, and a reference standard indicating a reference concentration of the at least one additional biomarker of heart failure in the biological sample.

42. The kit according to claim 40 for performing a method for providing a diagnosis or prognosis of a subject having or at risk of having renal disease, the kit further comprising at least one additional reagent capable of specifically binding at least one additional biomarker of renal disease in the biological sample to quantify the concentration of the at least one additional biomarker in the biological sample, and a reference standard indicating a reference concentration of the at least one additional biomarker of renal disease in the biological sample

43. The kit according to claim 40, wherein the at least one reagent comprises at least one antibody capable of specifically binding sFlt-1.
